(19) United States
(12) Patent Application Publication
(10) Pub. No.: US 2020/0327826 A1
(43) Pub. Date: Oct. 15, 2020

(54) SIMULATED SKIN SYSTEM

(71) Applicants: Stacie-Rae Anna Weir, Calgary, AB (CA); Samantha Rae Carniato, Errington, BC (CA)

(72) Inventors: Stacie-Rae Anna Weir, Calgary, AB (CA); Samantha Rae Carniato, Errington, BC (CA)

(21) Appl. No.: 16/384,604

(22) Filed: Apr. 15, 2019

(51) Int. Cl. G09B 23/30

(52) U.S. Cl. CPC G09B 23/30; A61B 5/444

(57) ABSTRACT

The present invention is a skin practice system provided for persons who work with or are training to work with professions requiring practice on skin surfaces.

### BACKGROUND OF THE INVENTION

Persons involved in various fields of endeavor relating to skin care, treatment, and enhancement, are often in a difficult situation relating to realistic practice devices. Although many artificial skin practice devices exists, a particular area that has been neglected relates to skin covering the breast. This would include persons involved in any type of endeavor. The range of applications would be medical professionals, therapists, and even tattoo artists. There are no realistic models that sufficiently and realistically portray skin covering the breast. The present invention has addressed this deficiency.

### SUMMARY OF THE INVENTION

In one embodiment, the present invention is a breast form with mastectomy scarring.

Additionally, the present invention provides for a practice skin (tattoo or non-tattoo) that shows any type of skin damage (we can license to others). Skin damage including, but not limited to, burns from any source as well as incisions for any reason, facelift, rash or acne, previous tattoo work, stretch mark, hypertrophic, atrophic, keloid, radiation scarring.

Additionally, there are no available Systems that provide for Any practice skin (tattoo or non-tattoo) of non-Caucasian base tone. The present invention contemplates configurations including Caucasian, Asian, Indian and African.

In one embodiment, the system of invention includes applying uniquely designed pigments or any other pigments and techniques.

The present invention provides:
1. a realistic practice medium;
2. can be any skin tone or detailed to show skin damage;
3. inexpensive yet realistic;
4. detailed to show skin damage an/or anomalies.

Device allows a practice medium for any who need it (doctors, tattooists, makeup artists, etc). By having realistic skin damage forms to practice on users can perfect techniques.

The present invention is the first to provide a practice skin in any color options other than a basic Caucasian tone.

In one embodiment, the present invention is a skin system for practice work, said system comprising;
a base material;
support material on top of said base;
skin on top of said support material;

In one embodiment, the base material is formed as two breasts. As understood, because this is a base material it is formed of suitable material having mass and density such that the system remains in place when being worked. In one embodiment, the base material weighs between 100-1000 grams. In the embodiment where the system is configures as a pair of breasts, as shown in the accompanying figures, each breast base is between 100-1000 grams.

In one embodiment, the support material is formed to provide support similar to breast tissue.

In one embodiment, the skin is removable.

In one embodiment, the skin is dyeable.

In one embodiment, the invention includes being supplied with a plurality of removable and changeable skins are selected from Caucasian, Oriental, African, Asian, or combinations thereof.

### BRIEF DESCRIPTION OF THE SEVERAL VIEWS OF THE DRAWINGS

FIG. 1 is a plan view according to one about them in the present invention.

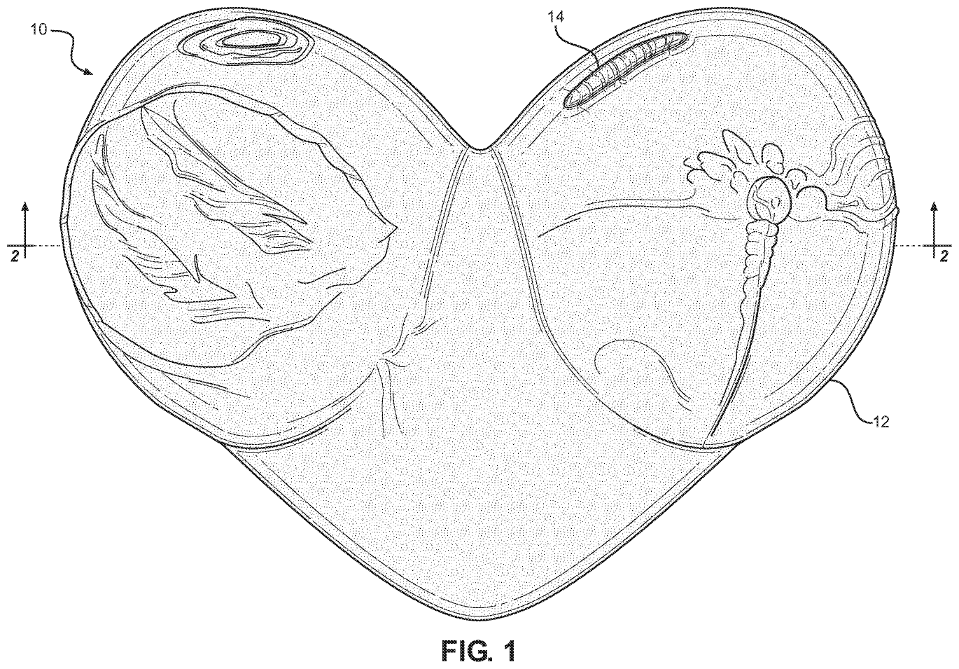

FIG. 2 is a cross-section view along sectional lines 2-2 from FIG. 1.

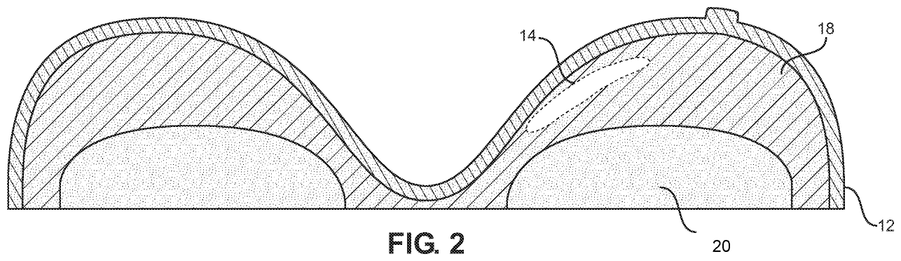

### DETAILED DESCRIPTION OF THE PREFERRED EMBODIMENT

As general interested in the figures, System 10 includes a pair of breast forms 20 surrounded by support material 18 that is covered by skin 12. In one embodiment, scar 14 is positioned in any region along skin 12 in order to simulate working with the breast having scar tissue.

Scar 14 represents damaged skin. There are no other practice skins have captured working with any type of scar tissue or skin damage

In one embodiment, the invention is a product that represents a breast form and shows common mastectomy and cancer damage.

It is further contemplate the present invention will provide configurations that will show different body parts and different types of damage, such as self-harm scars on the forearm.

In an embodiment depicting a forearm scar, the forearm self-harm scar product will also have applications in the medical field and beyond.

In one embodiment, the system is a configured as a phaloplasty for practice with male skin organ damage and anomalies.

In another embodiment, the system is configured for providing practice with skin damage and anomalies at and near the navel.

In another embodiment, the system is configured for providing practice with skin damage and anomalies at any one or more digits including fingers and toes.

In one embodiment, the present invention provides a configuration demonstrating specific damage, such as donor tissue, keloid, stretch marks, and thinness due to radiation.

As stated above, it is contemplated the present invention be provided in various configurations that will represent different ethnicities, which has never been done before. All current available practice skins only represent Caucasian skin tones.

The interior is provided with a pair of solid breast forms 20 for steady practice, and replaceable skins 12 in different colors, so students have a variety of applications to practice.

System 10, in one embodiment, is also configured to be tattooable, and can be used for decorative tattoo practice

Significant advantages of the present invention include:

Scarred practice skin for any application; and

Diverse ethnic representation in practice skin

In one embodiment, the presentation provides configurations having thick and thin parts consistent with a stress fracture in the dermis.

Puckering on inner bottom of the left side is almost consistent with a stitch seam created by the surgeon pulling the skin taut.

In one embodiment, skin 12 is configured depicting a thin spot that recreates a radiation burn which leaves the skin quite thin and tearable.

In one embodiment, skin 12 is configured depicting ‘lollypop’ scar consistent with the results of a breast lift, including moving the areola complex higher up on the breast.

In one embodiment, skin 12 is configured depicting a nipple protrusion consistent with results from a surgically created nipple protrusion, to teach practitioners how to work around any protrusions while keeping a consistent angle to achieves the correct depth of incision or needle reach.

Also demonstrated are common stitch patterns that have created lasting damage. These include, but are not limited to
puckers created by tight stitching and loose skin;
raised areas and different skin textures corresponding to use of donor tissue;
dimpled, corrugated, rippled, and raised areas corresponding to use of alloderm;
marbled scars consistent with necrosis, dying of tissue due to lack of blood flow;
dimpled skin to represent cellulite and/or atrophic scarring; and
raised skin to represent hypertrophic scarring as well as keloid scars;

In one embodiment, the president provides different textures and consistencies of silicon to represent the expressed tissue damage.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A skin system for practice work, said system comprising;
a base material;
support material on top of said base;
skin on top of said support material;

2. The system of claim 1, wherein said base material is formed as two breasts.

3. The system of claim 1, wherein said support material is formed to provide support similar to breast tissue.

4. The system of claim 1, wherein said skin is removable.

5. The system of claim 1, wherein said skin is dyeable.

6. The system of claim 1, wherein a plurality of skins are selected from Caucasian, Oriental, African, Asian, or combinations thereof are provided.

7. The system of claim 1, wherein said system is shaped and configured for providing practice with working with skin damage or anomalies.

8. The system of claim 1, wherein said system is shaped and configured for providing practice with working with skin damage or anomalies simulating one of breast, forearm, navel, fingers, or toes.
